(19) United States
(12) Patent Application Publication
(10) Pub. No.: US 2021/0355469 A1
(43) Pub. Date: Nov. 18, 2021

(54) ALPHA-AMYLASES WITH MUTATIONS THAT IMPROVE STABILITY IN THE PRESENCE OF CHELANTS

(71) Applicant: DANISCO US INC, Palo Alto, CA (US)

(72) Inventor: Jonathan LASSILA, Palo Alto, CA (US)

(21) Appl. No.: 17/284,669

(22) PCT Filed: Oct. 14, 2019
(86) PCT No.: PCT/US2019/056067

§ 371 Date: Apr. 12, 2021

Related U.S. Application Data
(60) Provisional application No. 62/745,070, filed on Oct. 12, 2018.

(51) Int. Cl. C12N 9/28; C11D 3/386; C12P 19/14

(52) U.S. Cl. CPC C12N 9/2417; C12P 19/14; C11D 3/386; C12Y 302/01001

(57) ABSTRACT

Disclosed are variant α-amylases having mutations that improve enzyme stability in the presence of chelants, methods of designing such variants, and methods of use, of the resulting variants. The variant α-amylases are particularly useful, for use in cleaning and desizing composition that include significant amounts of chelants.

### CROSS REFERENCE

The present application claims the benefit of U.S. Provisional Application Ser. No. 62/745,070, filed Oct. 12, 2018, which is hereby incorporated by reference in its entirety.

### TECHNICAL FIELD

Disclosed are variant α-amylases having mutations that improve enzyme stability in the presence of chelants, methods of designing such variants, and methods of use of the resulting variants. The variant α-amylases are particularly useful for use in cleaning and desizing composition that include significant amounts of chelants.

### BACKGROUND

Starch consists of a mixture of amylose (15-30% w/w) and amylopectin (70-85% w/w). Amylose consists of linear chains of α-1,4-linked glucose units having a molecular weight (MW) from about 60,000 to about 800,000. Amylopectin is a branched polymer containing α-1,6 branch points every 24-30 glucose units; its MW may be as high as 100 million.

α-amylases hydrolyze starch, glycogen, and related polysaccharides by cleaving internal α-1,4-glucosidic bonds at random. α-amylases, particularly from Bacilli, have been used for a variety of different purposes, including starch liquefaction and saccharification, textile desizing, starch modification in the paper and pulp industry, brewing, baking, production of syrups for the food industry, production of feed-stocks for fermentation processes, and in animal feed to increase digestability. These enzymes can also be used to remove starchy soils and stains during dishwashing and laundry washing.

Dishwashing and laundry detergent compositions, other hard cleaning compositions, and textile processing liquors, in particular but not exclusively, often contain significant amounts of chelants, primarily to reduce hard water deposits caused by the interaction of unpredictable levels of cations present in local water with components present in the cleaning or desizing compositions. Unfortunately, many of the most-preferred commercially available α-amylases rely on calcium-binding for stability and activity. Accordingly, the need exists to develop new α-amylases and ways to engineer α-amylases that are capable of a high level of performance and stability in the present of chelants.

### SUMMARY

The present compositions and methods relate to variant α-amylases having mutations that improve enzyme stability in the presence of chelants, methods of designing such variants, and methods of use of the resulting variants. Aspects and embodiments of the present compositions and methods are summarized in the following separately-numbered paragraphs:

1. In one aspect, a recombinant variant of a parental Family 13 α-amylase is provided, wherein the variant has a mutation (i) in the side chain of an amino acid residue that is not a ligand to a calcium or sodium ion, (ii) wherein the mutation is capable of altering the conformational freedom, the hydrogen bonding interactions, the pi stacking interactions, or the van der Waals interactions of the backbone loop that surrounds the Ca²⁺—Na⁺—Ca²⁺ site, and (iii) wherein the variant has increased stability in the presence of a predetermined amount of chelant compared to a the parental Family 13 α-amylase lacking the mutation.

2. In some embodiments of the variant of paragraph 1, the mutation is at an amino acid position selected from the group consisting of:

(i) E190, V206, H210, S244, and F245, using SEQ ID NO: 1 for numbering, or

(ii) E187, I203, H207, S241, and F242, using SEQ ID NO: 2 for numbering.

3. In some embodiments of the variant of paragraph 2, the mutation is a substitution selected from the group consisting of:

(i) E190P, V206T, V206Y, H210Q, S244C, S244D, S244H, S244N, S244E, S244F, S244V, S244L, S244Q and F245E, using SEQ ID NO: 1 for numbering, or

(ii) E187P, I203T, I203Y, H207Q, S241C, S241D, S241H, S241N, S241E, S241F, S241V, S241L, S241Q, and F242E, using SEQ ID NO: 2 for numbering.

4. In some embodiments, the variant of any of paragraphs 1-3 further comprises:

(i) a deletion or substitution at one or more residues corresponding to positions 181, 182, 183 and/or 184 in the amino acid sequence of SEQ ID NO: 1;

(ii) a deletion of residues 181 and 182 or 183 and 184 corresponding to the amino acid sequence of SEQ ID NO: 1;

(iii) a deletion of residues 178 and 179 or 180 and 181 corresponding to the amino acid sequence of SEQ ID NO: 2;

(iv) any single, multiple or combinatorial mutation(s) previously described in a Family 13 α-amylase; and/or

(v) an N-terminal and/or C-terminal truncation;

5. In some embodiments of the variant of any of paragraphs 1-4, the variant has at least 60%, 70%, 80%, or 90% amino acid sequence identity to the amino acid sequence of SEQ ID NO: 1 and/or SEQ ID NO: 2.

6. In another aspect, a detergent composition comprising the variant amylase of any of paragraphs 1-5 is provided, further comprising a chelating agent.

7. In another aspect, a composition for liquefying starch comprising the variant of any of paragraphs 1-5 is provided, further comprising a chelating agent.

8. In another aspect, a composition for desizing textiles comprising the variant of any of paragraphs 1-5 is provided, further comprising a chelating agent.

9. In another aspect, a composition for brewing or baking comprising the variant of any of paragraphs 1-5 is provided, further comprising a chelating agent.

10. In another aspect, a method for increasing the stability of a Family 13 α-amylase in the presence of a chelant is provided, comprising introducing to a parent Family 13 α-amylase a mutation (i) in the side chain of an amino acid residue that is not a ligand to a calcium or sodium ion, (ii) wherein the mutation is capable of altering the conformational freedom, the hydrogen bonding interactions, the pi stacking interactions, or the van der Waals interactions of the backbone loop that surrounds the Ca²⁺—Na⁺—Ca²⁺ site, and (iii) wherein the variant has increased stability in the presence of a predetermined amount of chelant compared to a the parental Family 13 α-amylase lacking the mutation.

11. In some embodiments of the method of paragraph 10, the mutation is at an amino acid position selected from the group consisting of:

(i) E190, V206, H210, S244, and F245, using SEQ ID NO: 1 for numbering, or

(ii) E187, I203, H207, S241, and F242, using SEQ ID NO: 2 for numbering.

12. In some embodiments of the method of paragraph 11, the mutation is a substitution selected from the group consisting of:

(i) E190P, V206T, V206Y, H210Q, S244C, S244D, S244H, S244N, S244E, S244F, S244V, S244L, S244Q and F245E, using SEQ ID NO: 1 for numbering, or

(ii) E187P, I203T, I203Y, H207Q, S241C, S241D, S241H, S241N, S241E, S241F, S241V, S241L, S241Q, and F242E, using SEQ ID NO: 2 for numbering.

13. In some embodiments of the method of any of paragraphs 10-12, the variant further comprises:

(i) a deletion or substitution at one or more residues corresponding to positions 181, 182, 183 and/or 184 in the amino acid sequence of SEQ ID NO: 1;

(ii) a deletion of residues 181 and 182 or 183 and 184 corresponding to the amino acid sequence of SEQ ID NO: 1;

(iii) a deletion of residues 178 and 179 or 180 and 181 corresponding to the amino acid sequence of SEQ ID NO: 2;

(iv) any single, multiple or combinatorial mutation(s) previously described in a Family 13 α-amylase; and/or

(v) an N-terminal and/or C-terminal truncation;

14. In some embodiments of the method of any of paragraphs 10-13, the variant has at least 60%, 70%, 80%, or 90% amino acid sequence identity to the amino acid sequence of SEQ ID NO: 1 and/or SEQ ID NO: 2.

15. In another aspect, a method for converting starch to oligosaccharides is provided, comprising contacting starch with effective amount of the variant α-amylase of any of paragraphs 1-5.

16. In another aspect, a method for removing a starchy stain or soil from a surface is provided, comprising contacting the surface with an effective amount of the variant α-amylase of any of paragraphs 1-5, or the composition of paragraph 7, and allowing the polypeptide to hydrolyze starch components present in the starchy stain to produce smaller starch-derived molecules that dissolve in the aqueous composition, thereby removing the starchy stain from the surface.

These and other aspects and embodiments of the compositions and methods will be apparent from the present description and drawings.

### BRIEF DESCRIPTION OF THE DRAWINGS

FIG. 1 shows models of two α-amylases highlighting with spheres the α-carbon positions for amino acid residues that, when mutated, provide a benefit in the presence of chelant. The BspAmy24 model is shown in light gray. The CspAmy2 model is shown in darker gray. Both molecules have an RG-deletion. Calcium and sodium ions are shown in black.

FIG. 2 highlights the location of a loop that surrounds the metal ion site and from which the majority of the metal ligands originate. The loop is shown in a thicker tube representation, whereas the rest of the structure is shown in a thinner wire representation. Amino acids in the BspAmy24 molecule are shown in light gray. Amino acids in the CspAmy2 molecule are shown in darker gray. Both molecules have an RG-deletion. Calcium and sodium ions are shown in spheres.

### DETAILED DESCRIPTION

Described are compositions and methods relating to variant α-amylases having mutations that improve enzyme stability in the presence of chelants, methods of designing such variants, and methods of use of the variants. Such variants are especially useful for cleaning starchy stains in laundry, dishwashing, textile processing (e.g., desizing), and other applications, in the presence of high levels of chelants, or in an environment of particularly soft water. These and other aspects of the compositions and methods are described in detail, below.

Prior to describing the various aspects and embodiments of the present compositions and methods, the following definitions and abbreviations are described.

### 1. Definitions and Abbreviations

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art. The following terms are defined, below, for clarity.

### 1.1. Abbreviations and Acronyms

The following abbreviations/acronyms have the following meanings unless otherwise specified:

DNA deoxyribonucleic acid

EC Enzyme Commission

GA glucoamylase

GH general hardness

HDL high density liquid detergent

HDD heavy duty powder detergent

HSG high suds granular detergent

HFCS high fructose corn syrup

IRS insoluble residual starch

kDa kiloDalton

MW molecular weight

MWU modified Wohlgemuth unit; 1.6×10⁻⁵ mg/MWU=unit of activity

NCBI National Center for Biotechnology Information

PI performance index

ppm parts per million, e.g., μg protein per gram dry solid

RCF relative centrifugal/centripetal force (i.e., x gravity)

sp. species

w/v weight/volume

w/w weight/weight

v/v volume/volume

wt % weight percent

° C. degrees Centigrade

H₂O water

dH₂O or DI deionized water

dIH₂O deionized water, Milli-Q filtration

g or gm grams

micrograms

mg milligrams

kg kilograms

μL and μl microliters

mL and ml milliliters

mm millimeters

μm micrometer

M molar

mM millimolar

micromolar

U units

sec seconds

min(s) minute/minutes

hr(s) hour/hours

ETOH ethanol

N normal

MWCO molecular weight cut-off

CAZy Carbohydrate-Active Enzymes database

WT wild-type

### 1.2. Definitions

The terms “amylase” or “amylolytic enzyme” refer to an enzyme that is, among other things, capable of catalyzing the degradation of starch. α-amylases are hydrolases that cleave the α-D-(1→4) O-glycosidic linkages in starch. Generally, α-amylases (EC 3.2.1.1; α-D-(1→4)-glucan glucanohydrolase) are defined as endo-acting enzymes cleaving α-D-(1→4) O-glycosidic linkages within the starch molecule in a random fashion yielding polysaccharides containing three or more (1-4)-α-linked D-glucose units. In contrast, the exo-acting amylolytic enzymes, such as β-amylases (EC 3.2.1.2; α-D-(1→4)-glucan maltohydrolase) and some product-specific amylases like maltogenic α-amylase (EC 3.2.1.133) cleave the polysaccharide molecule from the non-reducing end of the substrate. β-amylases, α-glucosidases (EC 3.2.1.20; α-D-glucoside glucohydrolase), glucoamylase (EC 3.2.1.3; α-D-(1→4)-glucan glucohydrolase), and product-specific amylases like the maltotetraosidases (EC 3.2.1.60) and the maltohexaosidases (EC 3.2.1.98) can produce malto-oligosaccharides of a specific length or enriched syrups of specific maltooligosaccharides.

The term “starch” refers to any material comprised of the complex polysaccharide carbohydrates of plants, comprised of amylose and amylopectin with the formula (C₆H₁₀O₅)ₓ, wherein X can be any number.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

In the case of the present α-amylases, “activity” refers to α-amylase activity, which can be measured as described, herein.

The term “performance benefit” refers to an improvement in a desirable property of a molecule. Exemplary performance benefits include, but are not limited to, increased hydrolysis of a starch substrate, increased grain, cereal or other starch substrate liquifaction performance, increased cleaning performance, increased thermal stability, increased detergent stability, increased storage stability, increased solubility, an altered pH profile, decreased calcium dependence, increased stability in the presence of chelants, increased specific activity, modified substrate specificity, modified substrate binding, modified pH-dependent activity, modified pH-dependent stability, increased oxidative stability, and increased expression. In some cases, the performance benefit is realized at a relatively low temperature. In some cases, the performance benefit is realized at relatively high temperature.

The terms “chelant” and “chelating agent” are used interchangeably to refer to a chemical compound capable of coordinating a metal ion, thereby preventing or reducing the possibility of the metal ion interacting with other components in a solution or suspension. Exemplary chelants are described, herein.

The terms “metal ligand” refers to atoms of an amino acid side chain, or main chain, that bind to metal, which may be found, for example, in the imidazole of histidine, thiol of cysteine, carboxylate of aspartate or glutamate, etc.

The terms “combinatorial variants” are variants comprising two or more mutations, e.g., 2, 3, 4, 5, 6, 7, 8, 9, 10, or more, substitutions, deletions, and/or insertions.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding an amylase is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptides, thereof, includes, but is not limited to, a culture broth containing secreted polypeptide expressed in a heterologous host cell.

The term “purified” refers to material (e.g., an isolated polypeptide or polynucleotide) that is in a relatively pure state, e.g., at least about 90% pure, at least about 95% pure, at least about 98% pure, or even at least about 99% pure.

The term “enriched” refers to material (e.g., an isolated polypeptide or polynucleotide) that is in about 50% pure, at least about 60% pure, at least about 70% pure, or even at least about 70% pure.

The terms “thermostable” and “thermostability,” with reference to an enzyme, refer to the ability of the enzyme to retain activity after exposure to an elevated temperature. The thermostability of an enzyme, such as an amylase enzyme, is measured by its half-life (t½) given in minutes, hours, or days, during which half the enzyme activity is lost under defined conditions. The half-life may be calculated by measuring residual α-amylase activity following exposure to (i.e., challenge by) an elevated temperature.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min., 1 hour).

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded, and may contain chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

“Hybridization” refers to the process by which one strand of nucleic acid forms a duplex with, i.e., base pairs with, a complementary strand, as occurs during blot hybridization techniques and PCR techniques. Stringent hybridization conditions are exemplified by hybridization under the following conditions: 65° C. and 0.1×SSC (where 1×SSC=0.15 M NaCl, 0.015 M Na3 citrate, pH 7.0). Hybridized, duplex nucleic acids are characterized by a melting temperature (Tm), where one half of the hybridized nucleic acids are unpaired with the complementary strand.

A “synthetic” molecule is produced by in vitro chemical or enzymatic synthesis rather than by an organism.

A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

The term “specific activity” refers to the number of moles of substrate that can be converted to product by an enzyme or enzyme preparation per unit time under specific conditions. Specific activity is generally expressed as units (U)/mg of protein.

As used herein, “water hardness” is a measure of the minerals (e.g., calcium and magnesium) present in water. The U.S. Geological Survey uses the following ranges of measurements to classify water into hard and soft water (Table 1):

TABLE 1
U.S. Geological Survey ranges of measurements to classify water.

| Description | Hardness (mg/L) | Hardness (mmol/L) |
|---|---|---|
| Soft | 0-60 | 0-0.60 |
| Moderately hard | 61-120 | 0.61-1.20 |
| Hard | 121-180 | 1.21-1.80 |
| Very hard | >181 | >1.81 |

A “swatch” is a piece of material such as a fabric that has a stain applied thereto. The material can be, for example, fabrics made of cotton, polyester or mixtures of natural and synthetic fibers. The swatch can further be paper, such as filter paper or nitrocellulose, or a piece of a hard material such as ceramic, metal, or glass. For α-amylases, the stain is starch based, but can include blood, milk, ink, grass, tea, wine, spinach, gravy, chocolate, egg, cheese, clay, pigment, oil, or mixtures of these compounds.

A “smaller swatch” or “micro swatch” is a section of the swatch that has been cut with a single hole punch device, or has been cut with a custom manufactured multiple-hole punch device, where the pattern of the multi-hole punch is matched to standard multi-well microtiter plates, or the section has been otherwise removed from the swatch. The swatch can be of textile, paper, metal, or other suitable material. The smaller swatch can have the stain affixed either before or after it is placed into the well of a 24-, 48- or 96-well microtiter plate. The smaller swatch can also be made by applying a stain to a small piece of material. For example, the smaller swatch can be a stained piece of fabric ⅝″ or 0.25″ or 5.5 mm in diameter. The custom manufactured punch is designed in such a manner that it delivers 96 swatches simultaneously to all wells of a 96-well plate. The device allows delivery of more than one swatch per well by simply loading the same 96-well plate multiple times. Multi-hole punch devices can be conceived of to deliver simultaneously swatches to any format plate, including but not limited to 24-well, 48-well, and 96-well plates. In another conceivable method, the soiled test platform can be a bead or tile made of metal, plastic, glass, ceramic, or another suitable material that is coated with the soil substrate. The one or more coated beads or tiles are then placed into wells of 96-, 48-, or 24-well plates or larger formats, containing suitable buffer and enzyme. In other conceivable methods, the stained fabric is exposed to enzyme by spotting enzyme solution onto the fabric, by wetting swatch attached to a holding device, or by immersing the swatch into a larger solution containing enzyme.

“Percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

Deletions are counted as non-identical residues, compared to a reference sequence.

The term “about” refers to ±15% to the referenced value.

### 2. Aspects and Embodiments of the Present Compositions and Methods

The following paragraphs describe in detail various aspects and embodiments of the present compositions and methods.

2.1. α-Amylase Variants Having Improved Resistance to Chelants

Screening was performed in two model CAZy Family 13 α-amylases to identify variants having improved stability in the presence of 5 mM etidronic acid (HEDP) chelant. Amino acid substitutions with improved chelant stability were found in a particular structural region of both proteins, which region is closely associated with the calcium binding site.

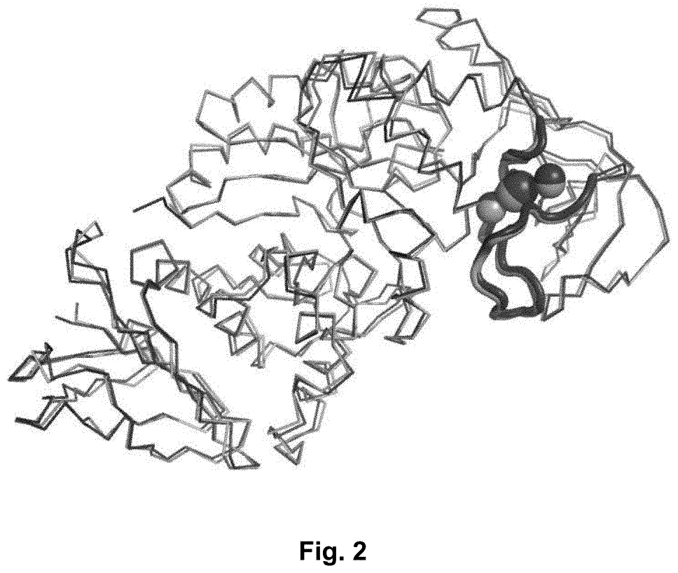

Without being limited to a theory, it is postulated that the loop formed by residues 185-210, corresponding to the amino acid sequence of BspAmy24 α-amylase (SEQ ID NO: 1), and to 182-207, corresponding to the amino acid sequence of CspAmy2 α-amylase (SEQ ID NO: 2), forms a cradle for the Ca²⁺—Na⁺—Ca²⁺ binding site (FIG. 2). This 185-210 loop, from which the majority of the metal ligands originate, surrounds the metal ion binding site and can be manipulated to modulate stability in the presence of chelants. It is reasoned that, in the presence of chelant, removal of the metal ions renders the 185-210 loop prone to deformation, thereby reducing the activation barrier for overall protein unfolding. Intramolecular interactions that stabilize the folded conformation of residues 185-210 and the positioning of this loop with respect to the spatially-adjacent secondary structure regions (i.e., residues 104-184, 211-230, 236-257, and 272-284) can stabilize the folded enzyme upon loss of ions to chelant.

Indeed, it was found that several substitutions that alter the conformational freedom of the 185-210 loop or the interactions of the 185-210 loop within the context of the adjacent protein structure regions provide large increases in stability to a common detergent chelant. These interactions were found to promote stability to chelant in two different α-amylases sharing amino acid sequence identity of less than 70%, indicating that the strategy is broadly applicable to CAZy Family 13 α-amylases.

Specifically, the present compositions and methods encompass amino acid mutations that result in an alteration in the side chain of an amino acid residue that is not a ligand to a calcium or sodium ion but is near the calcium site (i.e., has at least one atom within 12 Å of an atom of the Ca²⁺—Na⁺—Ca²⁺ metal site) and they are capable of altering the conformational freedom or the hydrogen bonding, pi stacking, or van der Waals interactions that stabilize the folded conformation of the aforementioned structural loop that surrounds the Ca²⁺—Na⁺—Ca²⁺ site.

One model α-amylase used to exemplify the present compositions and methods is an α-amylase from a Bacillus sp., herein referred to as “BspAmy24 α-amylase,” or simply, “BspAmy24.” The amino acid sequence of BspAmy24 α-amylase is shown, below, as SEQ ID NO: 1:

HHNGTNGTMM QYFEWHLPND GQHWNRLRND AANLKNLGIT
AVWIPPAWKG TSQNDVGYGA YDLYDLGEFN QKGTIRTKYG
TRSQLQSAIA SLQNNGIQVY GDVVMNHKGG ADGTEWVQAV
EVNPSNRNQE VTGEYTIEAW TKFDFPGRGN THSSFKWRWY
HFDGTDWDQS RQLNNRIYKF RGTGKAWDWE VDTENGNYDY
LMYADVDMDH PEVINELRRW GVWYTNTLNL DGFRIDAVKH
IKYSFTRDWL NHVRSTTGKN NMFAVAEFWK NDLGAIENYL
HKTNWNHSVF DVPLHYNLYN ASKSGGNYDM RQILNGTVVS
KHPIHAVTFV DNHDSQPAEA LESFVEAWFK PLAYALILTR
EQGYPSVFYG DYYGIPTHGV AAMKGKIDPI LEARQKYAYG
TQHDYLDHHN IIGWTREGNS AHPNSGLATI MSDGPGGSKW
MYVGRHKAGQ VWRDITGNRT GTVTINADGW GNFSVNGGSV
SIWVNK

A second model α-amylase used to exemplify the present compositions and methods is an α-amylase from a Cytophaga sp., herein referred to as “CspAmy2 α-amylase,” or simply, “CspAmy2”. The amino acid sequence of CspAmy2 α-amylase is shown, below, as SEQ ID NO: 2:

AATNGTMMQY FEWYVPNDGQ QWNRLRTDAP YLSSVGITAV
WTPPAYKGTS QADVGYGPYD LYDLGEFNQK GTVRTKYGTK
GELKSAVNTL HSNGIQVYGD VVMNHKAGAD YTENVTAVEV
NPSNRNQETS GEYNIQAWTG FNFPGRGTTY SNFKWQWFHF
DGTDWDQSRS LSRIFKFRGT GKAWDWEVSS ENGNYDYLMY
ADIDYDHPDV VNEMKKWGVW YANEVGLDGY RLDAVKHIKF
SFLKDWVDNA RAATGKEMFT VGEYWQNDLG ALNNYLAKVN
YNQSLFDAPL HYNFYAASTG GGYYDMRNIL NNTLVASNPT
KAVTLVENHD TQPGQSLEST VQPWFKPLAY AFILTRSGGY
PSVFYGDMYG TKGTTTREIP ALKSKIEPLL KARKDYAYGT
QRDYIDNPDV IGWTREGDST KAKSGLATVI TDGPGGSKRM
YVGTSNAGEI WYDLTGNRTD KITIGSDGYA TFPVNGGSVS
VWVQQ

In some embodiments, the variant α-amylase has at least 60%, at least 70%, at least 80%, at least 85%, at least 89%, at least 90%, at least 91%, at least 92%, at least 93%, at least 94%, at least 95%, at least 96%, at least 97%, at least 98%, or even at least 99% amino acid sequence identity to SEQ ID NO: 1 and/or SEQ ID NO: 2, excluding the wild-type BspAmy24 and CspAmy2 enzymes, and known variants, thereof.

It is known that many bacterial (and other) α-amylases share the same fold, and often benefit from the same mutations. In the present case, corresponding amino acid positions in other α-amylases can readily be identified by amino acid sequence alignment with BspAmy24 and CspAmy2, using Clustal W with default parameters. α-amylases in which the foregoing mutations are likely to produce a performance benefit include those having a similar fold and/or having 60% or greater amino acid sequence identity to any of the well-known Bacillus α-amylases (e.g., from B. licheniformis, B. stearothermophilus, B. amyloliquifaciens, Bacillus sp. SP722, and the like), Carbohydrate-Active Enzymes database (CAZy) Family 13 α-amylases, or any amylase that has heretofore been referred to by the descriptive term, “Termamyl-like.” The reader will appreciate that where an α-amylase naturally has a mutation listed above (i.e., where the wild-type α-amylase already comprised a residue identified as a mutation), then that particular mutation does not apply to that α-amylase. However, other described mutations may work in combination with the naturally occurring residue at that position.

### 2.2 Additional Mutations

In some embodiments, in addition to one or more of the mutations described above (e.g., in Section 2.1), the present α-amylases further include one or more mutations that provide a further performance or stability benefit. Exemplary performance benefits include but are not limited to increased hydrolysis of a starch substrate, increased grain, cereal or other starch substrate liquifaction performance, increased cleaning performance, increased thermal stability, increased storage stability, increased solubility, an altered pH profile, decreased calcium dependence, increased specific activity, modified substrate specificity, modified substrate binding, modified pH-dependent activity, modified pH-dependent stability, increased oxidative stability, and increased expression. In some cases, the performance benefit is realized at a relatively low temperature. In some cases, the performance benefit is realized at relatively high temperature.

In some embodiments, the present α-amylase variants additionally have at least one mutation in the calcium binding loop based on the work of Suzuki et al. (1989) J. Biol. Chem. 264:18933-938. Exemplary mutations include a deletion or substitution at one or more residues corresponding to positions 181, 182, 183 and/or 184 in SEQ ID NO: 1 and/or 2. In particular embodiments, the mutation corresponds to the deletion of 181 and 182 or 183 and 184 (using SEQ ID NO: 1 and/or 2 for numbering). Homologous residues in other α-amylases can be determined by structural alignment, or by primary structure alignment.

In some embodiments, the present α-amylase variants additionally have at least one mutation known to produce a performance, stability, or solubility benefit in other microbial α-amylases, including but not limited to those having a similar fold and/or having 60% or greater amino acid sequence identity to SEQ ID NO: 1 and/or 2, Carbohydrate-Active Enzymes database (CAZy) Family 13 amylases, or any amylase that has heretofore been referred to by the descriptive term, “Termamyl-like.” Amino acid sequence identity can be determined using Clustal W with default parameters.

The present α-amylases may include any number of conservative amino acid substitutions. Exemplary conservative amino acid substitutions are listed in Table 2.

TABLE 2
Conservative amino acid substitutions

| Amino Acid | Code | Replace with any of: |
|---|---|---|
| Alanine | A | D-Ala, Gly, β-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S—Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S—Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

It will be appreciated that some of the above mentioned conservative mutations can be produced by genetic manipulation, while others are produced by introducing synthetic amino acids into a polypeptide by genetic or other means.

The present amylase may also be derived from any of the above-described amylase variants by substitution, deletion or addition of one or several amino acids in the amino acid sequence, for example less than 10, less than 9, less than 8, less than 7, less than 6, less than 5, less than 4, less than 3, or even less than 2 substitutions, deletions or additions. Such variants should have the same activity as amylase from which they were derived. Particular deletions include N-terminal and/or C-terminal truncations of one or a few amino acid residues, for example, 1, 2, 3, 4, or 5 amino acid residues.

The present amylase may be “precursor,” “immature,” or “full-length,” in which case they include a signal sequence, or “mature,” in which case they lack a signal sequence. Mature forms of the polypeptides are generally the most useful. Unless otherwise noted, the amino acid residue numbering used herein refers to the mature forms of the respective amylase polypeptides. The present amylase polypeptides may also be truncated to remove the N or C-termini, so long as the resulting polypeptides retain amylase activity.

The present amylase may be a “chimeric,” “hybrid” or “domain swap” polypeptide, in that it includes at least a portion of a first amylase polypeptide, and at least a portion of a second amylase polypeptide. The present α-amylases may further include heterologous signal sequence, an epitope to allow tracking or purification, or the like. Exemplary heterologous signal sequences are from B. licheniformis amylase (LAT), B. subtilis (AmyE or AprE), and Streptomyces CelA.

2.3. Nucleotides Encoding Variant Amylase Polypeptides

In another aspect, nucleic acids encoding a variant amylase polypeptide are provided. The nucleic acid may encode a particular amylase polypeptide, or an amylase having a specified degree of amino acid sequence identity to the particular amylase.

In some embodiments, the nucleic acid encodes an amylase having at least 90%, at least 91%, at least 92%, at least 93%, at least 94%, at least 95%, at least 96%, at least 97%, at least 98%, or even at least 99% amino acid sequence identity to SEQ ID NO: 1 and/or 2. It will be appreciated that due to the degeneracy of the genetic code, a plurality of nucleic acids may encode the same polypeptide.

### 3. Exemplary Chelating Agents

A major issue concerning the formulation and use of cleaning compounds is water hardness, primarily due to the presence of calcium, magnesium, iron and manganese metal ions. Such metal ions interfere with the cleaning ability of surfactants and can result in significant amounts of precipitate with surfactants. Chelating agents (also called chelants) combine with metal ions to preclude precipitation with surfactants. Unfortunately, metal ions are frequently required for enzyme activity, making the formulation of detergent compositions an inevitable compromise.

Traditionally, the most common type of chelating agents used in industrial cleaning compounds has been phosphates. Phosphates have been banned in the US and Europe because they reenter the environment unchanged, even after sewage treatment, and cause oxygen depletion in waterways. Nonetheless, phosphates are still used in many countries and the present compositions and methods are fully compatible with phosphate-based chelants.

More environmentally friendly chelants, with which the present compositions and methods are compatible, include, but are not limited to, ethylene-diamine-tetraacetic acid (EDTA), diethylene triamine penta methylene phosphonic acid (DTPMP), hydroxy-ethane diphosphonic acid (HEDP), ethylenediamine N,N′-disuccinic acid (EDDS), methyl glycine diacetic acid (MGDA), glutamic acid N,N-diacetic acid (N,N-dicarboxymethyl glutamic acid, tetrasodium salt (GLDA), diethylene triamine penta acetic acid (DTPA), propylene diamine tetracetic acid (PDTA), 2-hydroxypyridine-N-oxide (HPNO), nitrilotriacetic acid (NTA), 4,5-dihydroxy-m-benzenedisulfonic acid, N-hydroxyethylethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP), citrate and gluconate (and any salts thereof) and derivatives of the aforementioned compounds.

### 4. Production of Variant α-Amylases

The present variant α-amylases can be produced in host cells, for example, by secretion or intracellular expression, using methods well-known in the art. Fermentation, separation, and concentration techniques are well known in the art and conventional methods can be used to prepare a concentrated, variant-α-amylase-polypeptide-containing solution.

For production scale recovery, variant α-amylase polypeptides can be enriched or partially purified as generally described above by removing cells via flocculation with polymers. Alternatively, the enzyme can be enriched or purified by microfiltration followed by concentration by ultrafiltration using available membranes and equipment. However, for some applications, the enzyme does not need to be enriched or purified, and whole broth culture can be lysed and used without further treatment. The enzyme can then be processed, for example, into granules.

### 5. Carbohydrate Processing Compositions and Uses Involving Variant α-Amylases

The present variants α-amylases are useful for a variety of carbohydrate processing applications that are well-known in the art. Such application may involve the use of chelants, including but not limited to those listed, herein, especially where local available water supplies are particularly hard. Exemplary applications include fuel ethanol production, syrup production and the production of other valuable biochemicals.

5.1. Preparation of Starch Substrates

Methods for preparing starch substrates for use in the processes disclosed herein are well known. Useful starch substrates may be obtained from, e.g., tubers, roots, stems, legumes, cereals or whole grain. More specifically, the granular starch may be obtained from corn, cobs, wheat, barley, rye, triticale, milo, sago, millet, cassava, tapioca, sorghum, rice, peas, bean, banana, or potatoes. Specifically contemplated starch substrates are corn starch and wheat starch. The starch from a grain may be ground or whole and includes corn solids, such as kernels, bran and/or cobs. The starch may also be highly refined raw starch or feedstock from starch refinery processes.

5.2. Gelatinization and Liquefaction of Starch

Gelatinization is generally performed simultaneously with, or followed by, contacting a starch substrate with an α-amylase, although additional liquefaction-inducing enzymes optionally may be added. In some embodiments, the starch substrate prepared as described above is slurried with water. Liquifaction may also be performed at or below the liquifaction temperatures, as in a “cold cook” or “no cook process.”

5.3. Saccharification

The liquefied starch can be saccharified into a syrup that is rich in lower DP (e.g., DP1+DP2) saccharides, using variant α-amylases, optionally in the presence of another enzyme(s). The exact composition of the products of saccharification depends on the combination of enzymes used, as well as the type of granular starch processed. Saccharification and fermentation may be performed simultaneously or in an overlapping manner (see, below).

5.4. Isomerization

The soluble starch hydrolysate produced by treatment with amylase can be converted into high fructose starch-based syrup (HFSS), such as high fructose corn syrup (HFCS). This conversion can be achieved using a glucose isomerase, particularly a glucose isomerase immobilized on a solid support.

5.5. Fermentation

The soluble starch hydrolysate, particularly a glucose rich syrup, can be fermented by contacting the starch hydrolysate with a fermenting organism. EOF products include metabolites, such as citric acid, lactic acid, succinic acid, monosodium glutamate, gluconic acid, sodium gluconate, calcium gluconate, potassium gluconate, itaconic acid and other carboxylic acids, glucono delta-lactone, sodium erythorbate, lysine and other amino acids, omega 3 fatty acid, butanol, isoprene, 1,3-propanediol and other biomaterials.

Ethanologenic microorganisms include yeast, such as Saccharomyces cerevisiae and bacteria, such as Zymomonas moblis, expressing alcohol dehydrogenase and pyruvate decarboxylase. Improved strains of ethanologenic microorganisms are known in the art. Commercial sources of yeast include ETHANOL RED® (LeSaffre); FERMAX™ (Martrex), THERMOSACC®, TRANSFERM® Yield+ and YP3™ (Lallemand); RED STAR® (Red Star); FERMIOL® (DSM Specialties); SUPERSTART® (Alltech); and SYNERXIA® and SYNERXIA® Thrive (DuPont Industrial Biosciences). Microorganisms that produce other metabolites, such as citric acid and lactic acid, by fermentation are also known in the art.

5.6. Carbohydrate Processing Compositions Comprising Variants α-Amylases and Additional Enzymes

The present variant α-amylases may be combined with a glucoamylase (EC 3.2.1.3), from e.g., Trichoderma, Aspergillus, Talaromyces, Clostridium, Fusarium, Thielavia, Thermomyces, Athelia, Humicola, Penicillium, Artomyces, Gloeophyllum, Pycnoporus, Steccherinum, Trametes etc. Suitable commercial glucoamylases, include AMG 200L; AMG 300 L; SAN™ SUPER and AMG™ E (Novozymes); OPTIDEX® 300 and OPTIDEX L-400 (DuPont Industrial Biosciences); AMIGASE™ and AMIGASE™ PLUS (DSM); G-ZYME® G900 (Enzyme Bio-Systems); and G-ZYME® G990 ZR.

Other suitable enzymes that can be used with amylase include phytase, protease, pullulanase, β-amylase, isoamylase, α-glucosidase, cellulase, xylanase, other hemicellulases, β-glucosidase, transferase, pectinase, lipase, cutinase, esterase, mannanase, redox enzymes, a different α-amylase, or a combination thereof.

Compositions comprising the present α-amylases may be aqueous or non-aqueous formulations, granules, powders, gels, slurries, pastes, etc., which may further comprise any one or more of the additional enzymes listed, herein, along with buffers, salts, preservatives, water, co-solvents, surfactants, and the like. Such compositions may work in combination with endogenous enzymes or other ingredients already present in a slurry, water bath, washing machine, food or drink product, etc., for example, endogenous plant (including algal) enzymes, residual enzymes from a prior processing step, and the like.

### 6. Compositions and Methods for Food and Feed Preparation

The present variant compositions and methods are also compatible with food and feed applications involving the use of chelants, including but not limited to those listed, herein. Such applications include the preparation of food products, animal feed and/or food/feed additives. An exemplary application, primarily for the benefit of humans, is baking.

### 7. Brewing Compositions

The present compositions and methods are also applicable to brewing applications involving the use of chelants, including but not limited to those listed, herein. While hard water is often desirable to produce certain styles and varieties of beers (or distilled products, thereof), it may be desirable to reduce the hardness of local water to enable the local production of other types and varieties of beer.

### 8. Textile Desizing Compositions

Also contemplated are the use of the present compositions and methods for treating fabrics (e.g., to desize a textile) in applications involving the use of chelants, including but not limited to those listed, herein, especially where local available water supplies are particularly hard. Fabric-treating methods are well known in the art (see, e.g., U.S. Pat. No. 6,077,316). The fabric can be treated with the solution under pressure.

### 9. Cleaning Compositions

An aspect of the present compositions and methods is a cleaning composition that includes chelants, including but not limited to those listed, herein as components. Such applications include, e.g., hand washing, laundry washing, dishwashing, and other hard-surface cleaning. Corresponding compositions include heavy duty liquid (HDL), heavy duty dry (HDD), and hand (manual) laundry detergent compositions, including unit dose format laundry detergent compositions, and automatic dishwashing (ADW) and hand (manual) dishwashing compositions, including unit dose format dishwashing compositions.

9.1. Overview

The present amylase polypeptides may be a component of a detergent composition comprising a chelants, as the only enzyme or with other enzymes including other amylolytic enzymes. It may be included in the detergent composition in the form of a non-dusting granulate, a stabilized liquid, or a protected enzyme.

The detergent composition may be in any useful form, e.g., as powders, granules, pastes, bars, or liquid. A liquid detergent may be aqueous, typically containing up to about 70% of water and 0% to about 30% of organic solvent. It may also be in the form of a compact gel type containing only about 30% water. The detergent composition comprises one or more surfactants, each of which may be anionic, nonionic, cationic, or zwitterionic. The detergent composition may additionally comprise one or more other enzymes, such as proteases, another amylolytic enzyme, mannanase, cutinase, lipase, cellulase, pectate lyase, perhydrolase, xylanase, peroxidase, and/or laccase in any combination.

Particular forms of detergent compositions for inclusion of the present α-amylase are described, below. Many of these composition can be provided in unit dose format for ease of use. Unit dose formulations and packaging are described in, for example, US20090209445A1, US20100081598A1, U.S. Pat. No. 7,001,878B2, EP1504994B1, WO2001085888A2, WO2003089562A1, WO2009098659A1, WO2009098660A1, WO2009112992A1, WO2009124160A1, WO2009152031A1, WO2010059483A1, WO2010088112A1, WO2010090915A1, WO2010135238A1, WO2011094687A1, WO2011094690A1, WO2011127102A1, WO2011163428A1, WO2008000567A1, WO2006045391A1, WO2006007911A1, WO2012027404A1, EP1740690B1, WO2012059336A1, U.S. Pat. No. 6,730,646B1, WO2008087426A1, WO2010116139A1 and WO2012104613A1.

9.2. Heavy Duty Liquid (HDL) Laundry Detergent Composition

Exemplary HDL laundry detergent compositions includes a detersive surfactant (10%-40% wt/wt), including an anionic detersive surfactant (selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof), and optionally non-ionic surfactant (selected from a group of linear or branched or random chain, substituted or unsubstituted alkyl alkoxylated alcohol, for example a C8-C18 alkyl ethoxylated alcohol and/or C6-C12 alkyl phenol alkoxylates), wherein the weight ratio of anionic detersive surfactant (with a hydrophilic index (HIc) of from 6.0 to 9) to non-ionic detersive surfactant is greater than 1:1. Suitable detersive surfactants also include cationic detersive surfactants (selected from a group of alkyl pyridinium compounds, alkyl quaternary ammonium compounds, alkyl quaternary phosphonium compounds, alkyl ternary sulphonium compounds, and/or mixtures thereof); zwitterionic and/or amphoteric detersive surfactants (selected from a group of alkanolamine sulpho-betaines); ampholytic surfactants; semi-polar non-ionic surfactants and mixtures thereof.

The composition may optionally include, a surfactancy boosting polymer consisting of amphiphilic alkoxylated grease cleaning polymers (selected from a group of alkoxylated polymers having branched hydrophilic and hydrophobic properties, such as alkoxylated polyalkylenimines in the range of 0.05 wt %-10 wt %) and/or random graft polymers (typically comprising of hydrophilic backbone comprising monomers selected from the group consisting of: unsaturated C1-C6 carboxylic acids, ethers, alcohols, aldehydes, ketones, esters, sugar units, alkoxy units, maleic anhydride, saturated polyalcohols such as glycerol, and mixtures thereof and hydrophobic side chain(s) selected from the group consisting of: C4-C25 alkyl group, polypropylene, polybutylene, vinyl ester of a saturated C1-C6 mono-carboxylic acid, C1-C6 alkyl ester of acrylic or methacrylic acid, and mixtures thereof.

The composition may include additional polymers such as soil release polymers (include anionically end-capped polyesters, for example SRP1, polymers comprising at least one monomer unit selected from saccharide, dicarboxylic acid, polyol and combinations thereof, in random or block configuration, ethylene terephthalate-based polymers and co-polymers thereof in random or block configuration, for example Repel-o-tex SF, SF-2 and SRP6, Texcare SRA100, SRA300, SRN100, SRN170, SRN240, SRN300 and SRN325, Marloquest SL), anti-redeposition polymers (0.1 wt % to 10 wt %, include carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof, vinylpyrrolidone homopolymer, and/or polyethylene glycol, molecular weight in the range of from 500 to 100,000 Da); cellulosic polymer (including those selected from alkyl cellulose, alkyl alkoxyalkyl cellulose, carboxyalkyl cellulose, alkyl carboxyalkyl cellulose examples of which include carboxymethyl cellulose, methyl cellulose, methyl hydroxyethyl cellulose, methyl carboxymethyl cellulose, and mixtures thereof) and polymeric carboxylate (such as maleate/acrylate random copolymer or polyacrylate homopolymer).

The composition may further include saturated or unsaturated fatty acid, preferably saturated or unsaturated C12-C24 fatty acid (0 wt % to 10 wt %); deposition aids (examples for which include polysaccharides, preferably cellulosic polymers, poly diallyl dimethyl ammonium halides (DADMAC), and co-polymers of DAD MAC with vinyl pyrrolidone, acrylamides, imidazoles, imidazolinium halides, and mixtures thereof, in random or block configuration, cationic guar gum, cationic cellulose such as cationic hydroxyethyl cellulose, cationic starch, cationic polyacrylamides, and mixtures thereof.

The composition may further include dye transfer inhibiting agents, examples of which include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof.

The composition preferably includes enzymes (generally about 0.01 wt % active enzyme to 0.03 wt % active enzyme) selected from α-amylases (including the present α-amylases and optionally pother α-amylases), proteases, lipases, cellulases, choline oxidases, peroxidases/oxidases, pectate lyases, mannanases, cutinases, laccases, phospholipases, lysophospholipases, acyltransferases, perhydrolases, arylesterases, and any mixture thereof. The composition may include an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

The composition optionally includes silicone or fatty-acid based suds suppressors; hueing dyes, calcium and magnesium cations, visual signaling ingredients, anti-foam (0.001 wt % to about 4.0 wt %), and/or structurant/thickener (0.01 wt % to 5 wt %, selected from the group consisting of diglycerides and triglycerides, ethylene glycol distearate, microcrystalline cellulose, cellulose based materials, microfiber cellulose, biopolymers, xanthan gum, gellan gum, and mixtures thereof).

The composition can be any liquid form, for example a liquid or gel form, or any combination thereof. The composition may be in any unit dose form, for example a pouch.

9.3. Heavy Duty Dry/Solid (HDD) Laundry Detergent Composition

Exemplary HDD laundry detergent compositions includes a detersive surfactant, including anionic detersive surfactants (e.g., linear or branched or random chain, substituted or unsubstituted alkyl sulphates, alkyl sulphonates, alkyl alkoxylated sulphate, alkyl phosphates, alkyl phosphonates, alkyl carboxylates and/or mixtures thereof), non-ionic detersive surfactant (e.g., linear or branched or random chain, substituted or unsubstituted C8-C18 alkyl ethoxylates, and/or C6-C12 alkyl phenol alkoxylates), cationic detersive surfactants (e.g., alkyl pyridinium compounds, alkyl quaternary ammonium compounds, alkyl quaternary phosphonium compounds, alkyl ternary sulphonium compounds, and mixtures thereof), zwitterionic and/or amphoteric detersive surfactants (e.g., alkanolamine sulpho-betaines), ampholytic surfactants, semi-polar non-ionic surfactants, and mixtures thereof; builders including phosphate free builders (for example zeolite builders examples which include zeolite A, zeolite X, zeolite P and zeolite MAP in the range of 0 wt % to less than 10 wt %), phosphate builders (for example sodium tri-polyphosphate in the range of 0 wt % to less than 10 wt %), citric acid, citrate salts and nitrilotriacetic acid, silicate salt (e.g., sodium or potassium silicate or sodium meta-silicate in the range of 0 wt % to less than 10 wt %, or layered silicate (SKS-6)); carbonate salt (e.g., sodium carbonate and/or sodium bicarbonate in the range of 0 wt % to less than 80 wt %); and bleaching agents including photobleaches (e.g., sulfonated zinc phthalocyanines, sulfonated aluminum phthalocyanines, xanthenes dyes, and mixtures thereof) hydrophobic or hydrophilic bleach activators (e.g., dodecanoyl oxybenzene sulfonate, decanoyl oxybenzene sulfonate, decanoyl oxybenzoic acid or salts thereof, 3,5,5-trimethy hexanoyl oxybenzene sulfonate, tetraacetyl ethylene diamine-TAED, nonanoyloxybenzene sulfonate-NOBS, nitrile quats, and mixtures thereof), sources of hydrogen peroxide (e.g., inorganic perhydrate salts examples of which include mono or tetra hydrate sodium salt of perborate, percarbonate, persulfate, perphosphate, or persilicate), preformed hydrophilic and/or hydrophobic peracids (e.g., percarboxylic acids and salts, percarbonic acids and salts, perimidic acids and salts, peroxymonosulfuric acids and salts, and mixtures thereof), and/or bleach catalysts (e.g., imine bleach boosters (examples of which include iminium cations and polyions), iminium zwitterions, modified amines, modified amine oxides, N-sulphonyl imines, N-phosphonyl imines, N-acyl imines, thiadiazole dioxides, perfluoroimines, cyclic sugar ketones, and mixtures thereof, and metal-containing bleach catalysts (e.g., copper, iron, titanium, ruthenium, tungsten, molybdenum, or manganese cations along with an auxiliary metal cations such as zinc or aluminum.

The composition preferably includes enzymes, e.g., proteases, amylases, lipases, cellulases, choline oxidases, peroxidases/oxidases, pectate lyases, mannanases, cutinases, laccases, phospholipases, lysophospholipases, acyltransferase, perhydrolase, arylesterase, and any mixture thereof.

The composition may optionally include additional detergent ingredients including perfume microcapsules, starch encapsulated perfume accord, hueing agents, additional polymers, including fabric integrity and cationic polymers, dye-lock ingredients, fabric-softening agents, brighteners (for example C.I. Fluorescent brighteners), flocculating agents, chelating agents, alkoxylated polyamines, fabric deposition aids, and/or cyclodextrin.

9.4. Automatic Dishwashing (ADW) Detergent Composition

Exemplary ADW detergent composition includes non-ionic surfactants, including ethoxylated non-ionic surfactants, alcohol alkoxylated surfactants, epoxy-capped poly(oxyalkylated) alcohols, or amine oxide surfactants present in amounts from 0 to 10% by weight; builders in the range of 5-60%, homopolymers and copolymers of poly-carboxylic acids and their partially or completely neutralized salts, monomeric polycarboxylic acids and hydroxycarboxylic acids and their salts in the range of 0.5% to 50% by weight; sulfonated/carboxylated polymers in the range of about 0.1% to about 50% by weight to provide dimensional stability; drying aids in the range of about 0.1% to about 10% by weight (e.g., polyesters, especially anionic polyesters, optionally together with further monomers with 3 to 6 functionalities—typically acid, alcohol or ester functionalities which are conducive to polycondensation, polycarbonate-, polyurethane- and/or polyurea-polyorganosiloxane compounds or precursor compounds, thereof, particularly of the reactive cyclic carbonate and urea type); silicates in the range from about 1% to about 20% by weight (including sodium or potassium silicates for example sodium disilicate, sodium meta-silicate and crystalline phyllosilicates); inorganic bleach (e.g., perhydrate salts such as perborate, percarbonate, perphosphate, persulfate and persilicate salts) and organic bleach (e.g., organic peroxyacids, including diacyl and tetraacylperoxides, especially diperoxydodecanedioc acid, diperoxytetradecanedioc acid, and diperoxyhexadecanedioc acid); bleach activators (i.e., organic peracid precursors in the range from about 0.1% to about 10% by weight); bleach catalysts (e.g., manganese triazacyclononane and related complexes, Co, Cu, Mn, and Fe bispyridylamine and related complexes, and pentamine acetate cobalt(III) and related complexes); metal care agents in the range from about 0.1% to 5% by weight (e.g., benzatriazoles, metal salts and complexes, and/or silicates); enzymes in the range from about 0.01 to 5.0 mg of active enzyme per gram of automatic dishwashing detergent composition (e.g., proteases, amylases, lipases, cellulases, choline oxidases, peroxidases/oxidases, pectate lyases, mannanases, cutinases, laccases, phospholipases, lysophospholipases, acyltransferase, perhydrolase, arylesterase, and mixtures thereof); and enzyme stabilizer components (e.g., oligosaccharides, polysaccharides, and inorganic divalent metal salts).

9.5. Additional Enzymes

Any of the chelant-containing cleaning compositions described, herein, may include any number of additional enzymes. In general, the enzyme(s) should be compatible with the selected detergent, (e.g., with respect to pH-optimum, compatibility with other enzymatic and non-enzymatic ingredients, and the like), and the enzyme(s) should be present in effective amounts. The following enzymes are provided as examples.

Suitable proteases include those of animal, vegetable or microbial origin. Chemically modified or protein engineered mutants are included, as well as naturally processed proteins. The protease may be a serine protease or a metalloprotease, an alkaline microbial protease, a trypsin-like protease, or a chymotrypsin-like protease. Examples of alkaline proteases are subtilisins, especially those derived from Bacillus, e.g., subtilisin Novo, subtilisin Carlsberg, subtilisin 309, subtilisin 147, and subtilisin 168 (see, e.g., WO 89/06279). Exemplary proteases include but are not limited to those described in WO199523221, WO199221760, WO2008010925, WO20100566356, WO2011072099, WO201113022, WO2011140364, WO2012151534, WO2015038792, WO2015089441, WO2015089447, WO2015143360, WO2016001449, WO2016001450, WO2016061438, WO2016069544, WO2016069548, WO2016069552, WO 2016069557, WO2016069563, WO2016069569, WO2016087617, WO2016087619, WO2016145428, WO2016174234, WO2016183509, WO2016202835, WO2016205755, US 2008/0090747, U.S. Pat. Nos. 5,801,039, 5,340,735, 5,500,364, 5,855,625, RE 34,606, U.S. Pat. Nos. 5,955,340, 5,700,676, 6,312,936, 6,482,628, 8,530,219, U.S. Provisional App. Nos. 62/331,282, 62/343,618, 62/351,649, 62/437,171, 62/437,174, and 62/437,509, and PCT App. Nos. PCT/CN2017/076749 and, as well as metalloproteases described in WO 2007/044993, WO 2009/058303, WO 2009/058661, WO 2014/071410, WO 2014/194032, WO 2014/194034, WO 2014/194054, and WO 2014/194117.

Exemplary commercial proteases include, but are not limited to MAXATASE, MAXACAL, MAXAPEM, OPTICLEAN®, OPTIMASE®, PROPERASE®, PURAFECT®, PURAFECT® OXP, PURAMAX®, EXCELLASE®, PREFERENZ™ proteases (e.g., P100, P110, P280), EFFECTENZ™ proteases (e.g., P1000, P1050, P2000), EXCELLENZ™ proteases (e.g., P1000), ULTIMASE®, and PURAFAST (DuPont Industrial Biosciences); ALCALASE®, ALCALASE® ULTRA, BLAZE®, BLAZE® EVITY®, BLAZE® EVITY® 16L, CORONASE®, SAVINASE®, SAVINASE® ULTRA, SAVINASE® EVITY®, SAVINASE® EVERTS®, PRIMASE, DURAZYM, POLARZYME®, OVOZYME®, KANNASE®, LIQUANASE®, EVERTS®, NEUTRASE®, PROGRESS UNO®, RELASE® and ESPERASE® (Novozymes); BLAP™ and BLAP™ variants (Henkel); LAVERGY™ PRO 104 L (BASF), and KAP® (B. alkalophilus subtilisin) (Kao). Suitable proteases include naturally occurring proteases or engineered variants specifically selected or engineered to work at relatively low temperatures.

Suitable lipases include those of bacterial or fungal origin. Chemically modified, proteolytically modified, or protein engineered mutants are included. Examples of useful lipases include but are not limited to lipases from Humicola (synonym Thermomyces), e.g., from H. lanuginosa (T. lanuginosus) (see e.g., EP 258068 and EP 305216), from H. insolens (see e.g., WO 96/13580); a Pseudomonas lipase (e.g., from P. alcaligenes or P. pseudoalcaligenes; see, e.g., EP 218 272), P. cepacia (see e.g., EP 331 376), P. stutzeri (see e.g., GB 1,372,034), P. fluorescens, Pseudomonas sp. strain SD 705 (see e.g., WO 95/06720 and WO 96/27002), P. wisconsinensis (see e.g., WO 96/12012); a Bacillus lipase (e.g., from B. subtilis; see e.g., Dartois et al. (1993) Biochemica et Biophysica Acta 1131:253-360), B. stearothermophilus (see e.g., JP 64/744992), or B. pumilus (see e.g., WO 91/16422). Additional lipase variants contemplated for use in the formulations include those described for example in: WO 92/05249, WO 94/01541, WO 95/35381, WO 96/00292, WO 95/30744, WO 94/25578, WO 95/14783, WO 95/22615, WO 97/04079, WO 97/07202, EP 407225, and EP 260105.

Exemplary commercial lipases include, but are not limited to M1 LIPASE, LUMA FAST, and LIPOMAX (DuPont Industrial Biosciences); LIPEX®, LIPOCLEAN®, LIPOLASE® and LIPOLASE® ULTRA (Novozymes); and LIPASE P (Amano Pharmaceutical Co. Ltd).

Polyesterases: Suitable polyesterases can be included in the composition, such as those described in, for example, WO 01/34899, WO 01/14629, and U.S. Pat. No. 6,933,140.

The present compositions can be combined with other amylases, including other α-amylases. Such a combination is particularly desirable when different α-amylases demonstrate different performance characteristics and the combination of a plurality of different α-amylases results in a composition that provides the benefits of the different α-amylases. Other α-amylases include commercially available α-amylases, such as but not limited to STAINZYME®, NATALASE®, DURAMYL®, TERMAMYL®, FUNGAMYL® and BAN™ (Novo Nordisk A/S and Novozymes A/S); RAPIDASE®, POWERASE®, PURASTAR®, and PREFERENZ™ (from DuPont Industrial Biosciences). Exemplary α-amylases are described in WO9418314A1, US20080293607, WO2013063460, WO10115028, WO2009061380A2, WO2014099523, WO2015077126A1, WO2013184577, WO2014164777, WO9510603, WO9526397, WO9623874, WO9623873, WO9741213, WO9919467, WO0060060, WO0029560, WO9923211, WO9946399, WO0060058, WO0060059, WO9942567, WO0114532, WO02092797, WO0166712, WO0188107, WO0196537, WO0210355, WO2006002643, WO2004055178, and WO9813481.

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed for example in U.S. Pat. Nos. 4,435,307; 5,648,263; 5,691,178; 5,776,757; and WO 89/09259. Exemplary cellulases contemplated for use are those having color care benefit for the textile. Examples of such cellulases are cellulases described in for example EP 0495257, EP 0531372, WO 96/11262, WO 96/29397, and WO 98/08940. Other examples are cellulase variants, such as those described in WO 94/07998; WO 98/12307; WO 95/24471; PCT/DK98/00299; EP 531315; U.S. Pat. Nos. 5,457,046; 5,686,593; and 5,763,254. Exemplary cellulases include those described in WO2005054475, WO2005056787, U.S. Pat. Nos. 7,449,318, 7,833,773, 4,435,307; EP 0495257; and U.S. Provisional Appl. Nos. 62/296,678 and 62/435,340. Exemplary commercial cellulases include, but are not limited to, CELLUCLEAN®, CELLUZYME®, CAREZYME®, CAREZYME® PREMIUM, ENDOLASE®, and RENOZYME® (Novozymes); REVITALENZ®100, REVITALENZ® 200/220 and REVITALENZ® 2000 (DuPont Industrial Biosciences); and KAC-500(B) (Kao Corporation).

Exemplary mannanases include, but are not limited to, those of bacterial or fungal origin, such as, for example, as is described in WO2016007929; U.S. Pat. Nos. 6,566,114, 6,602,842, and 6,440,991; and International Appl. Nos. PCT/US2016/060850 and PCT/US2016/060844. Exemplary mannanases include, but are not limited to, those of bacterial or fungal origin, such as, for example, as is described in WO2016007929; U.S. Pat. Nos. 6,566,114, 6,602,842, and 6,440,991; and International Appl. Nos. PCT/US2016/060850 and PCT/US2016/060844.

Suitable peroxidases/oxidases contemplated for use in the compositions include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include for example GUARDZYME™ (Novo Nordisk A/S and Novozymes A/S).

The detergent composition can also comprise 2,6-β-D-fructan hydrolase, which is effective for removal/cleaning of biofilm present on household and/or industrial textile/laundry.

The detergent enzyme(s) may be included in a detergent composition by adding separate additives containing one or more enzymes, or by adding a combined additive comprising all of these enzymes. A detergent additive, i.e. a separate additive or a combined additive, can be formulated, e.g., as a granulate, a liquid, a slurry, and the like. Exemplary detergent additive formulations include but are not limited to granulates, in particular non-dusting granulates, liquids, in particular stabilized liquids or slurries.

The detergent composition may be in any convenient form, e.g., a bar, a tablet, a powder, a granule, a paste, or a liquid. A liquid detergent may be aqueous, typically containing up to about 70% water, and 0% to about 30% organic solvent. Compact detergent gels containing about 30% or less water are also contemplated.

Numerous exemplary detergent formulations to which the present α-amylases can be added (or is in some cases are identified as a component of) are described in WO2013063460. These include commercially available unit dose detergent formulations/packages such as PUREX® UltraPacks (Henkel), FINISH® Quantum (Reckitt Benckiser), CLOROX™ 2 Packs (Clorox), OxiClean Max Force Power Paks (Church & Dwight), TIDE® Stain Release, CASCADE® ActionPacs, and TIDE® Pods (Procter & Gamble), PS.

9.6. Methods of Assessing Amylase Activity in Detergent Compositions

Numerous α-amylase cleaning assays are known in the art, including swatch and micro-swatch assays. The appended Examples describe only a few such assays.

In order to further illustrate the compositions and methods, and advantages thereof, the following specific examples are given with the understanding that they are illustrative rather than limiting.

All references cited herein are herein incorporated by reference in their entirety for all purposes. In order to further illustrate the compositions and methods, and advantages thereof, the following specific examples are given with the understanding that they are illustrative rather than limiting.

### EXAMPLES

### Example 1: Strain and Sample Separation

DNA sequences encoding the proteins of interest were obtained using conventional gene synthesis methods. A signal peptide for secretion and additional 5′ and 3′ sequences for amplification and subcloning were introduced using standard PCR amplification techniques. Alternatively, entire synthetic genes can be commerically produced. Standard procedures were used to insert these DNA sequences into bacterial vectors for integration and secretion in Bacillus subtilis or Bacillus licheniformis cells. The constructs were verified by DNA sequencing. Transformed cells were grown for 68-hr in suitable expression medium.

Cells were separated from protein-containing supernatant by centrifugation followed by filtration through 0.45 μm membranes (EMD Millipore). In some cases, additional purification was achieved through ion exchange chromatography using a Phenyl Sepharose 6 Fast Flow resin (GE Healthcare). Protein concentration was determined by high performance liquid chromatography (HPLC) and absorbance at 280 nm.

### Example 2: Stability of Variants

The relative chelant stability of the described engineered variants was evaluated by measurements based on the relative loss of activity upon incubation in a chelant solution at elevated temperatures. In brief, enzymes were diluted into a chelant solution to a concentration of approximately 1-5 ppm. The chelant solution consisted of 50 mM CAPS, 0.005% Tween-80, and 5 mM etidronic acid (HEDP) adjusted to pH 10.5. The enzyme-containing solutions were stressed by heating in a thermocycler for between 4 and 10 minutes at between 65 and 85° C. Samples of the enzyme in test solutions were taken both before and after stressing the solution at elevated temperature. The amylase activity present in the samples was evaluated using the Amylase HR assay (Megazyme). All variants included the well-known “RG-deletion” (i.e., “ΔRG”),” referring to residues R181 and G182 of BspAmy24 and R178 and G179 of CspAmy2. Mutations that showed improvement in the two α-amylases are shown in Table 4, with positions aligned by row in the two molecules. Several mutations were found to improve chelant stability in both molecules, despite the two α-amylases having amino acid sequence identity of less than 70%.

TABLE 4
Mutations that improved chelant stability
of BspAmy24 and CspAmy2 variants.

| Mutations in wt | | | Mutations in wt | | |
|---|---|---|---|---|---|
| BspAmy24 ΔRG | | Residual | CspAmy2 ΔRG | | Residual |
| ΔRG | Std | Activity | ΔRG | Std | Activity |
| numbering | numbering | (%) | numbering | numbering | (%) |
| none | — | 41 | none | — | 30 |
| E188P | E190P | nd | E185P | E187P | 65 |
| V204T | V206T | 71 | I201T | I203T | nd |
| V204Y | V206Y | 74 | I201Y | I203Y | 59 |
| H208Q | H210Q | 68 | H205Q | H207Q | nd |
| S242C | S244C | 97 | S239C | S241C | 55 |
| S242D | S244D | 71 | S239D | S241D | 91 |
| S242H | S244H | 77 | S239H | S241H | 64 |
| S242N | S244N | 67 | S239N | S241N | 56 |
| S242E | S244E | 100 | S239E | S241E | 78 |
| S242F | S244F | 95 | S239F | S241F | 58 |
| S242V | S244V | 65 | S239V | S241V | 56 |
| S242L | S244L | 84 | S239L | S241L | 55 |
| S242Q | S244Q | 84 | S239Q | S241Q | 65 |
| F243E | F245E | 93 | F240E | F242E | 63 |

### Example 3: Structural Analysis of Variants

Homology models of BspAmy24 and CspAmy2 α-amylase were constructed as follows. The amino acid sequence of BspAmy24 (SEQ ID NO: 1) or CspAmy2 (SEQ ID NO: 2) was used as a query in MOE (Chemical Computing Group, Montreal, CA) to search the Protein Data Bank (see e.g., Berman, H. E. et al. (2000) Nuc. Acids Res. 28:235-42). The Bacillus licheniformis α-amylase (1BLI) was the top public hit for both searches. The “homology model” function, with all default parameters, was used to create a model for each enzyme. An x-ray diffraction crystal structure was also determined for a BspAmy24 variant α-amylase and a CspAmy2 variant α-amylase. These experimental structures closely matched the homology models and supported the analysis performed with the homology models.

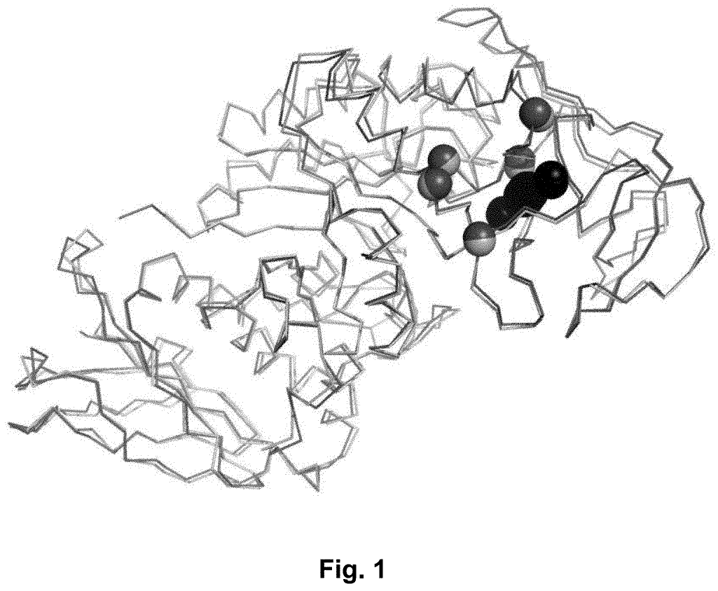

The positions of amino acids from Table 4 are shown in the structural alignment of the α-amylase models in FIG. 1. The α carbons for these five positions are shown for each amylase as spheres. Amino acids in the BspAmy24 α-amylase molecule (with the herein described RG-deletion) are shown in light gray. Amino acids in the CspAmy2 α-amylase molecule (again with the RG-deletion) are shown in darker gray. Calcium and sodium ions are shown in black. As seen in the Figure, the positions from Table 4 show a close structural alignment in the two molecules.

Structural modeling also indicates that mutations in these positions are likely to alter interactions that stabilize the conformation of the 185-210 loop and its positioning within the folded protein structure. The loop in positions 185-210 (BspAmy24 numbering) surrounds the Ca²⁺—Na⁺—Ca²⁺ metal site and contains the majority of ligands to these metal ions (FIG. 2). The amino acid mutations listed in Table 4 may alter the interactions that stabilize the 185-210 loop either as a result of being within the loop or as a result of being capable of interacting with the loop as indicated in Table 5.

TABLE 5
Locations of amino acid positions within the structure

| Position in | Position in | |
|---|---|---|
| BspAmy24 | CspAmy2 | Location within structure |
| E190 | E187 | Within the 185-210 loop |
| V206 | I203 | Within the 185-210 loop |
| H210 | H207 | Within the 185-210 loop |
| S244 | S241 | Capable of interacting with |
| | | the 185-210 loop |
| F245 | F242 | Capable of interacting with |
| | | the 185-210 loop |

Further observations from structural modeling suggest specific types of interactions of the 185-210 loop that may be altered upon mutation, given the locations and conformations of the amino acids from Table 4 and their surrounding structural environments. The E190P/E187P mutation would stabilize the folded structure of the loop by restricting the conformational freedom of the loop to the more limited phi and psi angles available to the proline side chain. Mutations in position 206/203 will change the van der Waals and hydrophobic packing interactions with nearby regions of protein structure. Steric changes can move the backbone that hydrogen bonds with an adjacent strand at that position (BspAmy24-Asn106). Mutation to tyrosine could create a new hydrogen bond and/or pi stacking with adjacent residues. The H210Q/H207Q mutations could create new hydrogen bonds with the backbone at BspAmy24-Glu212 or BspAmy24-Tyr160 or with the BspAmy24-Lys185 side chain. Mutations in position 244/241 could generate new hydrogen bonding interactions with the 185-210 loop and also will alter van der Waals interactions that Ser makes with BspAmy24-Lys242, which is within feasible hydrogen bonding geometry of three positions on the 185-210 loop. Mutations of Phe at position 245/242 are expected to alter van der Waals and pi stacking interactions with residues on the 185-210 loop, BspAmy24-Met208/CspAmy24-Tyr205. Mutation to Glu may also alter potential hydrogen bonds at loop residues BspAmy24-Asp209, BspAmy24-Asp188, and BspAmy24-Met208. Note that any of these interactions may result in small local adjustments of the conformation of the 185-210 loop, while at the same time stabilizing the overall folded structure of the loop and thus increasing the overall protein stability in the presence of chelant.

What is claimed is:

1. A recombinant variant of a parental Family 13 α-amylase, wherein the variant has a mutation (i) in the side chain of an amino acid residue that is not a ligand to a calcium or sodium ion, (ii) wherein the mutation is capable of altering the conformational freedom, the hydrogen bonding interactions, the pi stacking interactions, or the van der Waals interactions of the backbone loop that surrounds the Ca²⁺—Na⁺—Ca²⁺ site, and (iii) wherein the variant has increased stability in the presence of a predetermined amount of chelant compared to a the parental Family 13 α-amylase lacking the mutation.

2. The variant of claim 1, wherein the mutation is at an amino acid position selected from the group consisting of:
(i) E190, V206, H210, S244, and F245, using SEQ ID NO: 1 for numbering, or
(ii) E187, I203, H207, S241, and F242, using SEQ ID NO: 2 for numbering.

3. The variant of claim 2, wherein the mutation is a substitution selected from the group consisting of:
(i) E190P, V206T, V206Y, H210Q, S244C, S244D, S244H, S244N, S244E, S244F, S244V, S244L, S244Q and F245E, using SEQ ID NO: 1 for numbering, or
(ii) E187P, I203T, I203Y, H207Q, S241C, S241D, S241H, S241N, S241E, S241F, S241V, S241L, S241Q, and F242E, using SEQ ID NO: 2 for numbering.

4. The variant of any of claims 1-3, further comprising:
(i) a deletion or substitution at one or more residues corresponding to positions 181, 182, 183 and/or 184 in the amino acid sequence of SEQ ID NO: 1;
(ii) a deletion of residues 181 and 182 or 183 and 184 corresponding to the amino acid sequence of SEQ ID NO: 1;
(iii) a deletion of residues 178 and 179 or 180 and 181 corresponding to the amino acid sequence of SEQ ID NO: 2;
(iv) any single, multiple or combinatorial mutation(s) previously described in a Family 13 α-amylase; and/or
(v) an N-terminal and/or C-terminal truncation;

5. The variant of any of claims 1-4, wherein the variant has at least 60%, 70%, 80%, or 90% amino acid sequence identity to the amino acid sequence of SEQ ID NO: 1 and/or SEQ ID NO: 2.

6. A detergent composition comprising the variant amylase of any of claims 1-5, further comprising a chelating agent.

7. A composition for liquefying starch comprising the variant of any of claims 1-5, further comprising a chelating agent.

8. A composition for desizing textiles comprising the variant of any of claims 1-5, further comprising a chelating agent.

9. A composition for brewing or baking comprising the variant of any of claims 1-5, further comprising a chelating agent.

10. A method for increasing the stability of a Family 13 α-amylase in the presence of a chelant, comprising introducing to a parent Family 13 α-amylase a mutation (i) in the side chain of an amino acid residue that is not a ligand to a calcium or sodium ion, (ii) wherein the mutation is capable of altering the conformational freedom, the hydrogen bonding interactions, the pi stacking interactions, or the van der Waals interactions of the backbone loop that surrounds the Ca²⁺—Na⁺—Ca²⁺ site, and (iii) wherein the variant has increased stability in the presence of a predetermined amount of chelant compared to a the parental Family 13 α-amylase lacking the mutation.

11. The method of claim 10, wherein the mutation is at an amino acid position selected from the group consisting of:
(i) E190, V206, H210, S244, and F245, using SEQ ID NO: 1 for numbering, or
(ii) E187, I203, H207, S241, and F242, using SEQ ID NO: 2 for numbering.

12. The method of claim 11, wherein the mutation is a substitution selected from the group consisting of:
(i) E190P, V206T, V206Y, H210Q, S244C, S244D, S244H, S244N, S244E, S244F, S244V, S244L, S244Q and F245E, using SEQ ID NO: 1 for numbering, or
(ii) E187P, I203T, I203Y, H207Q, S241C, S241D, S241H, S241N, S241E, S241F, S241V, S241L, S241Q, and F242E, using SEQ ID NO: 2 for numbering.

13. The method of any of claims 10-12, wherein the variant further comprises:
(i) a deletion or substitution at one or more residues corresponding to positions 181, 182, 183 and/or 184 in the amino acid sequence of SEQ ID NO: 1;
(ii) a deletion of residues 181 and 182 or 183 and 184 corresponding to the amino acid sequence of SEQ ID NO: 1;
(iii) a deletion of residues 178 and 179 or 180 and 181 corresponding to the amino acid sequence of SEQ ID NO: 2;
(iv) any single, multiple or combinatorial mutation(s) previously described in a Family 13 α-amylase; and/or
(v) an N-terminal and/or C-terminal truncation;

14. The method of any of claims 10-13, wherein the variant has at least 60%, 70%, 80%, or 90% amino acid sequence identity to the amino acid sequence of SEQ ID NO: 1 and/or SEQ ID NO: 2.

15. A method for converting starch to oligosaccharides, comprising contacting starch with effective amount of the variant α-amylase of any of claims 1-5.

16. A method for removing a starchy stain or soil from a surface, comprising contacting the surface with an effective amount of the variant α-amylase of any of claims 1-5, or the composition of claim 7, and allowing the polypeptide to hydrolyze starch components present in the starchy stain to produce smaller starch-derived molecules that dissolve in the aqueous composition, thereby removing the starchy stain from the surface.
